(19) United States
(12) Patent Application Publication
(10) Pub. No.: US 2023/0103667 A1
(43) Pub. Date: Apr. 6, 2023

(54) METHODS AND COMPOSITIONS FOR PREVENTING AND TREATING RETINAL DAMAGE IN GLAUCOMA

(71) Applicants: G3P, INC, Burlington, MA (US); MASSACHUSETTS EYE AND EAR INFIRMARY, Boston, MA (US); THE BRIGHAM AND WOMEN'S HOSPITAL, INC., BOSTON, MA (US)

(72) Inventors: Pieter Muntendam, Boxford, MA (US); Millca A. Margeta, Boston, MA (US); David A. Sola-Del Valle, Boston, MA (US); Oleg Butovsky, Boston, MA (US)

(21) Appl. No.: 17/960,501

(22) Filed: Oct. 5, 2022

Related U.S. Application Data
(63) Continuation of application No. PCT/US2021/026113, filed on Apr. 7, 2021.
(60) Provisional application No. 63/006,176, filed on Apr. 7, 2020.

(51) Int. Cl. A61K 31/732; A61K 31/7004; A61K 9/00; A61P 27/06; A61K 31/7016

(52) U.S. Cl. CPC A61K 31/732; A61K 31/7004; A61K 9/0048; A61P 27/06; A61K 31/7016

(57) ABSTRACT

The invention provides methods, compositions, and kits using a galectin-3 inhibitor to prevent and treat retinal nerve damage in a subject suffering from glaucoma.

### CROSS REFERENCE TO RELATED APPLICATION

This application is a continuation of International (PCT) Patent Application Ser. No. PCT/US2021/026113, filed Apr. 7, 2021, which claims the benefit of and priority to United States Provisional Application No. 63/006,176, filed Apr. 7, 2020; the contents of each of which are hereby incorporated by reference in their entirety.

### FIELD OF THE INVENTION

The invention provides methods, compositions, and kits using a galectin-3 inhibitor to prevent and treat retinal nerve damage in a subject suffering from glaucoma.

### BACKGROUND

Galectin-3 is a protein belonging to a specific sub-family of carbohydrate binding proteins (lectins) that recognize beta-galactosides. Galectins possess a carbohydrate recognition domain (CRD). The CRDs of various galectins differ in amino acid sequence outside of the conserved residues and this mediates specificity to different glycan ligands between galectins. Galectin-3 has both intracellular functions and extracellular functions and is actively secreted via a non-canonical pathway into the extracellular space and into the circulation. Binding of carbohydrates to the CRD results in modulation of galectin-3 activity in-vitro and in-vivo. Carbohydrate binding to the CRD and the resulting inhibition of galectin-3 is recognized as a potential therapeutic modality.

Glaucoma is a leading cause of blindness and often characterized by a buildup of fluid within the eye which can cause an increase in intraocular pressure (TOP). The increase in TOP can damage to nerves in the retina, resulting in cellular death and vision loss. In a healthy eye, the ocular fluid containing nutrients and that bathes the eye is continuously drained and replenished. However, in a subject suffering from glaucoma, the ocular fluid either does not drain properly or is created in excess, resulting in an increase in intraocular pressure.

The need exists for methods of preventing and treating retinal nerve damage in patients suffering from glaucoma. The present invention addresses this need and provides other related advantages.

### SUMMARY

The invention provides methods, compositions, and kits using a galectin-3 inhibitor to prevent and treat retinal nerve damage in a subject suffering from glaucoma. The galectin-3 inhibitor may be, for example, a carbohydrate, such as a pectin. The retina contains nerve fibers, including nerve endings from the optical nerve. While not being bound to a particular theory, the galectin-3 inhibitor is believed to prevent and/or treat damage to nerves in the retina that can otherwise occur due in part to elevated intraocular pressure in patients suffering from glaucoma. In this way, the galectin-3 inhibitor prevents and/or treats retinal nerve damage.

One aspect of the invention provides a method of treating retinal nerve damage in a subject suffering from glaucoma, wherein the method comprises administering to a subject in need thereof a therapeutically effective amount of a galectin-3 inhibitor to treat the retinal nerve damage. Another aspect of the invention provides a method of prophylaxis of retinal nerve damage in a subject suffering from glaucoma, wherein the method comprises administering to a subject in need thereof an effective amount of a galectin-3 inhibitor. Another aspect of the invention provides a method of reducing the risk of retinal nerve damage in a subject suffering from glaucoma, wherein the method comprises administering to a subject in need thereof an effective amount of a galectin-3 inhibitor to reduce the risk of retinal nerve damage in the subject. The methods may provide particular benefit in subjects featuring elevated intraocular pressure, elevated blood plasma levels of galectin-3, and/or elevated intraocular levels of galectin-3.

Compositions for use in the methods are provided, along with medical kits containing materials and instructions for implementing the method.

### BRIEF DESCRIPTION OF THE DRAWINGS

FIG. 1 is a graph showing the amount of fragments per kilobase of transcript per million mapped reads (FPKM) in different types of subjects, as described in more detail in Example 1.

FIG. 2 is an illustration showing immunohistochemistry for microglia, as described in more detail in Example 1.

FIG. 3 is an illustration showing immunohistochemistry for microglia, as described in more detail in Example 2.

FIG. 4 is an illustration of images from flatmounted retinas, as described in more detail in Example 3.

FIG. 5 is a graph showing observed amounts of retinal ganglions cells (RGC), as described in more detail in Example 3.

### DETAILED DESCRIPTION OF THE INVENTION

The invention provides methods, compositions, and kits using a galectin-3 inhibitor to prevent and treat retinal nerve damage in a subject suffering from glaucoma, such as retinal nerve damage resulting from elevated intraocular pressure. The retina contains nerve fibers, including nerve endings from the optical nerve. While not being bound to a particular theory, the galectin-3 inhibitor is believed to prevent and/or treat damage to nerves in the retina that can otherwise occur due in part to elevated intraocular pressure in patients suffering from glaucoma. In this way, the galectin-3 inhibitor prevents and/or treats retinal nerve damage. The methods and compositions provide particular benefits to patients exhibiting elevated intraocular pressure, elevated blood plasma levels of galectin-3, and/or elevated intraocular levels of galectin-3. Various aspects of the invention are set forth below in sections; however, aspects of the invention described in one particular section are not to be limited to any particular section.

### Definitions

To facilitate an understanding of the present invention, a number of terms and phrases are defined below.

The terms “a,” “an” and “the” as used herein mean “one or more” and include the plural unless the context is inappropriate

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results. Unless specified otherwise, an effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, or disorder, or ameliorating a symptom thereof. As used herein, the term “preventing” refers to delaying or precluding onset of the condition, disease, or disorder.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin in Remington's Pharmaceutical Sciences, 15th Ed., Mack Publ. Co., Easton, Pa. [1975].

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls. Certain numerical values herein are modified by the term about. In certain embodiments, about a stated value is within ±10% of the stated value; also provided are embodiments that are within ±9%, ±8%, ±7%, ±6%, ±5%, ±4%, ±3%, ±2%, or ±1% of the stated value.

### I. Therapeutic Methods

The invention provides methods for treating retinal nerve damage in a patient suffering from glaucoma, methods of prophylaxis of retinal damage in a subject suffering from glaucoma, and methods for reducing the risk of retinal nerve damage in a patient suffering from glaucoma. The methods may be characterized according to, for example, the identity of the galectin-3 inhibitor, the dosing regimen, and preferred patient populations. Various aspects and embodiments of the therapeutic methods are described in the sections below. The sections are arranged for convenience and information in one section is not to be limited to that section, but may be applied to methods in other sections.

### A. First Method

One aspect of the invention provides a method of treating retinal nerve damage in a subject suffering from glaucoma. The method comprises administering to a subject in need thereof a therapeutically effective amount of a galectin-3 inhibitor to treat the retinal nerve damage.

The method may be characterized according to, for example, the magnitude of reduction in the volume of the retinal nerve damage. In certain embodiments, the method achieves at least a 25% reduction in the volume of retinal nerve damage. In certain embodiments, the method achieves at least a 50% reduction in the volume retinal nerve damage. In certain embodiments, the method achieves at least a 25% reduction in the volume of retinal nerve damage compared to the average volume of retinal nerve damage in a subject having glaucoma and not having received the galectin-3 inhibitor. In certain embodiments, the method achieves at least a 50% reduction in the volume of retinal nerve damage compared to the volume of retinal nerve damage in a subject having glaucoma and not having received the galectin-3 inhibitor. In certain embodiments, the method achieves at least a 90% reduction in the volume of retinal nerve damage compared to the average volume of retinal nerve damage in a subject having glaucoma and not having received the galectin-3 inhibitor. The method may be further characterized according to additional exemplary features described below.

### B. Second Method

Another aspect of the invention provides a method of prophylaxis of retinal damage in a subject suffering from glaucoma. The method comprises administering to a subject in need thereof an effective amount of a galectin-3 inhibitor.

The method may be characterized according to, for example, the magnitude of reduction in the rate of progression of the retinal nerve damage. In certain embodiments, the method achieves at least a 25% reduction in the rate of progression of retinal nerve damage compared to the average rate of progression of retinal nerve damage in a subject having glaucoma and not having received the galectin-3 inhibitor. In certain embodiments, the method achieves at least a 50% reduction in the rate of progression of retinal nerve damage compared to the average rate of progression of retinal nerve damage in a subject having glaucoma and not having received the galectin-3 inhibitor. In certain embodiments, the method achieves at least a 90% reduction in the rate of progression of retinal nerve damage compared to the average rate of progression of retinal nerve damage in a subject having glaucoma and not having received the galectin-3 inhibitor. The method may be further characterized according to additional exemplary features described below.

### C. Third Method

Another aspect of the invention provides a method of reducing the risk of retinal nerve damage in a subject suffering from glaucoma. The method comprises administering to a subject in need thereof an effective amount of a galectin-3 inhibitor to reduce the risk of retinal nerve damage in the subject.

The method may be further characterized according to, for example, the magnitude of the reduction in risk of retinal nerve damage in a subject suffering from glaucoma. In certain embodiments, the method produces at least a 5%, 10%, 20%, 30%, 40%, 50%, 60%, 70%, 80%, 90%, or 95% reduction in the risk of retinal nerve damage. In certain embodiments, the method achieves at least a 25% reduction in risk of retinal nerve damage. In certain embodiments, the method achieves at least a 50% reduction in risk of retinal nerve damage. In certain embodiments, the method achieves at least a 90% reduction in risk of retinal nerve damage.

The method may be further characterized according to additional exemplary features described below.

### D. Additional Exemplary Features of the First, Second, and Third Therapeutic Methods

The First, Second, and Third Therapeutic Methods described herein may be further characterized according to, for example, the identity of the galectin-3 inhibitor, the dosing regimen, preferred patient populations, and other features described herein below. A more thorough description of such features is provided below. The invention embraces all permutations and combinations of these features, where appropriate.

1. Galectin-3 Inhibitors

The method may be characterized according to the identity of the galectin-3 inhibitor. For example, in certain embodiments, the galectin-3 inhibitor is a carbohydrate, protein, lipid, nucleic acid, or small organic compound. In certain embodiments, the galectin-3 inhibitor comprises a carbohydrate. In certain embodiments, the galectin-3 inhibitor comprises a polysaccharide. In certain embodiments, the galectin-3 inhibitor is a carbohydrate. In certain embodiments, the galectin-3 inhibitor is a polysaccharide.

In certain embodiments, the galectin-3 inhibitor comprises a pectin. In certain embodiments, the galectin-3 inhibitor is a pectin. Pectins are polysaccharides derived from plant cell walls, especially from apple and citrus fruits. A pectin used may be a full-length pectin or may be a pectin fragment. In certain embodiments, the pectin fragment may be purified according to procedures described in the literature. The pectin may be characterized according to its molecular weight. In certain embodiments, the pectin has a molecular weight in the range of from about 50 kDa to about 150 kDa, from about 60 kDa to about 130 kDa, from about 50 kDa to about 100 kDa, from about 30 kDa to about 60 kDa, from about 10 kDa to about 50 kDa, from about 10 kDa to about 30 kDa, from about 5 kDa to about 20 kDa, or from about 1 kDa to about 10 kDa.

In certain embodiments, the polysaccharide is a pumpkin pectin. In certain embodiments, the polysaccharide is an apple pectin. In certain embodiments, the polysaccharide is a citrus fruit pectin. In certain embodiments, the polysaccharide is a sugar beet pectin. In certain embodiments, the polysaccharide is a pear pectin. In certain embodiments, the polysaccharide is a potato pectin. In certain embodiments, the polysaccharide is a carrot pectin.

In certain embodiments, the galectin-3 inhibitor comprises a polysaccharide isolated from a plant material. In certain embodiments, the plant material is a member of the genus Cucurbita. In certain embodiments, the polysaccharide is isolated from C. moschata, C. argyrosperma, C. fwifolia, C. maxima, or C. pepo.

In certain embodiments, the polysaccharide comprises galactose. In certain embodiments, the galectin-3 inhibitor is galactose. In certain embodiments, the polysaccharide comprises a rhamnogalacturonan I (RG-I) domain. In certain embodiments, the RG-I domain comprises β-D-galactan, a-L-arabinofuranosyl, or combinations thereof. In certain embodiments, the polysaccharide comprises a homogalacturonan (HG) domain.

In certain embodiments, the polysaccharide has a molecular weight of about 5 kDa to about 70 kDa. In certain embodiments, the polysaccharide has a molecular weight of about 20 kDa to about 30 kDa. In certain embodiments, the polysaccharide has a molecular weight of about 20 kDa to about 25 kDa. In certain embodiments, the polysaccharide has a molecular weight of about 5 kDa to about 25 kDa. In certain embodiments, the polysaccharide has a molecular weight of about 17 kDa to about 23 kDa. In certain embodiments, the molecular weight of the polysaccharide is about 17.5 kDa. In certain embodiments, the galectin-3 inhibitor comprises or is a polysaccharide described in PCT Application Publication WO 2019/143924A1, the entirety of which is incorporated by reference herein.

In certain embodiments, the galectin-3 inhibitor comprises Modified Citrus Pectin (MCP). In certain embodiments, the galectin-3 inhibitor is MCP. MCP is different from other pectins, as it is modified from organic citrus pectin to reduce the molecular weight of the pectin molecule, such as to between about 10 kDa and about 30 kDa or between about 5 kDa and about 20 kDa.

In certain embodiments, the galectin-3 inhibitor is a pectic compound. Pectic compounds are derived from pectins, where a substantial portion of the pectin backbone has been removed. In certain embodiments, the galectin-3 inhibitor comprises a mixture of pectic fragments.

In certain embodiments, the galectin-3 inhibitor comprises a pectin-derived moiety.

In certain embodiments, the galectin-3 inhibitor comprises an artificial polysaccharide. In certain embodiments, the galectin-3 inhibitor is an artificial polysaccharide. In certain embodiments, the artificial polysaccharide is selected from GR-MD-02 and GM-CT-01 (Davanat™).

In certain embodiments, the polysaccharide is modified with one or more non-naturally occurring chemical moieties. In certain embodiments, the polysaccharide is given one or more modifications concurrent with or subsequent to isolation from a plant material. In certain embodiments, the one or more modifications include alkylation, amidation, quaternization, thiolation, sulfation, oxidation, chain elongation, e.g., cross-linking, grafting, etc., depolymerization by chemical, physical, or biological processes including enzymatic process, etc., or combinations thereof.

In certain embodiments, the galectin-3 inhibitor comprises a chemically modified polysaccharide. In certain embodiments, the galectin-3 inhibitor is a chemically modified polysaccharide. In certain embodiments, the chemically modified polysaccharide is TD139.

In certain embodiments, the polysaccharide has a galectin-3 binding affinity greater than that of potato galactan. In certain embodiments, the polysaccharide inhibits galectin-3 activity at concentrations of the polysaccharide below 2 mM. In certain embodiments, the polysaccharide inhibits galectin-3 activity at concentrations of the polysaccharide at about 1.26 mM.

In certain embodiments, the galectin-3 inhibitor comprises an oligosaccharide. In certain embodiments, the galectin-3 inhibitor is an oligosaccharide. In certain embodiments, the oligosaccharide is a neo-glycan. In certain embodiments, the oligosaccharide is N-acetyllactosamine. In certain embodiments, the oligosaccharide is a derivative of N-acetyllactosamine. In certain embodiments, the oligosaccharide is N,N-diacetyllactosamine.

In certain embodiments, the galectin-3 inhibitor comprises a protein, antibody, galectin binding protein (GBP) interaction fusion protein, peptide aptamer, Avimer, Fab, sFv, Adnectin, ligand, nucleic acid, or lipid. In certain embodiments, the galectin-3 inhibitor comprises an antibody, galectin binding protein (GBP) interaction fusion protein, peptide aptamer, Avimer, Fab, sFv, Adnectin, ligand, or nucleic acid.

In certain embodiments, the galectin-3 inhibitor comprises a protein. In certain embodiments, the galectin-3 inhibitor comprises an antibody, galectin binding protein (GBP) interaction fusion protein, peptide aptamer, Avimer, Fab, sFv, Adnectin, or ligand.

In certain embodiments, the galectin-3 inhibitor comprises an antibody. In certain embodiments, the galectin-3 inhibitor comprises a primary, secondary, monoclonal, polyclonal, human, humanized, or chimeric antibody. In certain embodiments, the galectin-3 inhibitor comprises a primary antibody. In certain embodiments, the galectin-3 inhibitor comprises a secondary antibody. In certain embodiments, the galectin-3 inhibitor comprises a monoclonal or polyclonal antibody. In certain embodiments, the galectin-3 inhibitor comprises a monoclonal antibody. In certain embodiments, the galectin-3 inhibitor comprises a polyclonal antibody. In certain embodiments, the galectin-3 inhibitor comprises a human antibody. In certain embodiments, the galectin-3 inhibitor comprises a humanized antibody. In certain embodiments, the galectin-3 inhibitor comprises chimeric antibody.

In certain embodiments, the galectin-3 inhibitor comprises antibody 87B5. In certain embodiments, the galectin-3 inhibitor is antibody 87B5. In certain embodiments, the galectin-3 inhibitor comprises antibody M3/38. In certain embodiments, the galectin-3 inhibitor is antibody M3/38.

In certain embodiments, the galectin-3 inhibitor comprises an antibody fragment. In certain embodiments, the galectin-3 inhibitor comprises a single chain Fv antibody (sFv). In certain embodiments, the galectin-3 inhibitor comprises an antigen-binding fragment (Fab).

In certain embodiments, the galectin-3 inhibitor comprises a galectin binding protein (GBP) interaction fusion protein. In certain embodiments, the galectin-3 inhibitor comprises a peptide aptamer. In certain embodiments, the galectin-3 inhibitor comprises an Avimer. In certain embodiments, the galectin-3 inhibitor comprises an Adnectin. In certain embodiments, the galectin-3 inhibitor comprises an A1-41-41BODY® ligand.

In certain embodiments, the galectin-3 inhibitor comprises a nucleic acid. In certain embodiments, the galectin-3 inhibitor comprises DNA. In certain embodiments, the galectin-3 inhibitor comprises RNA. In certain embodiments, the galectin-3 inhibitor comprises a nucleotide aptamer.

In certain embodiments, the galectin-3 inhibitor comprises a lipid. In certain embodiments, the galectin-3 inhibitor comprises a membrane lipid.

In certain embodiments, the galectin-3 inhibitor is a protein, nucleic acid, or lipid.

In certain embodiments, the galectin-3 inhibitor is a protein, antibody, galectin binding protein (GBP) interaction fusion protein, peptide aptamer, Avimer, Fab, sFv, Adnectin, ligand, nucleic acid, or lipid. In certain embodiments, the galectin-3 inhibitor is an antibody, galectin binding protein (GBP) interaction fusion protein, peptide aptamer, Avimer, Fab, sFv, Adnectin, ligand, or nucleic acid. In certain embodiments, the galectin-3 inhibitor is an antibody. In certain embodiments, the galectin-3 inhibitor comprises an antibody.

In certain embodiments, the galectin-3 inhibitor is a protein. In certain embodiments, the galectin-3 inhibitor is an antibody, galectin binding protein (GBP) interaction fusion protein, peptide aptamer, Avimer, Fab, sFv, Adnectin, or ligand.

In certain embodiments, the galectin-3 inhibitor is an antibody. In certain embodiments, the galectin-3 inhibitor is a primary, secondary, monoclonal, polyclonal, human, humanized, or chimeric antibody. In certain embodiments, the galectin-3 inhibitor is a primary antibody. In certain embodiments, the galectin-3 inhibitor is a secondary antibody. In certain embodiments, the galectin-3 inhibitor is a monoclonal or polyclonal antibody. In certain embodiments, the galectin-3 inhibitor is a monoclonal antibody. In certain embodiments, the galectin-3 inhibitor is a polyclonal antibody. In certain embodiments, the galectin-3 inhibitor is a human antibody. In certain embodiments, the galectin-3 inhibitor is a humanized antibody. In certain embodiments, the galectin-3 inhibitor is chimeric antibody.

In certain embodiments, the galectin-3 inhibitor is an antibody fragment. In certain embodiments, the galectin-3 inhibitor is a single chain Fv antibody (sFv). In certain embodiments, the galectin-3 inhibitor is an antigen-binding fragment (Fab).

In certain embodiments, the galectin-3 inhibitor is a galectin binding protein (GBP) interaction fusion protein. In certain embodiments, the galectin-3 inhibitor is a peptide aptamer. In certain embodiments, the galectin-3 inhibitor is an Avimer. In certain embodiments, the galectin-3 inhibitor is an Adnectin. In certain embodiments, the galectin-3 inhibitor is an AFFIBODY® ligand.

In certain embodiments, the galectin-3 inhibitor is a nucleic acid. In certain embodiments, the galectin-3 inhibitor is DNA. In certain embodiments, the galectin-3 inhibitor is RNA. In certain embodiments, the galectin-3 inhibitor is a nucleotide aptamer.

In certain embodiments, the galectin-3 inhibitor is a lipid. In certain embodiments, the galectin-3 inhibitor is a membrane lipid.

In certain embodiments, the galectin-3 inhibitor is a small organic molecule.

2. Dosing Regimen

In certain embodiments, the method may be characterized based on the amount of galectin-3 inhibitor being administered and/or frequency with which the galectin-3 inhibitor is administered to the subject. The galectin-3 inhibitor can be dosed, for example, based on the weight of the subject or as a fixed dose. In certain embodiments, the galectin-3 inhibitor is administered 1, 2, or 3 times per day. In certain embodiments, each administration of galectin-3 inhibitor provides from about 0.1 g to about 0.5 g, from about 0.5 to about 1.0, from about 1.0 to about 2.0 g, or from about 2 to about 3 g of galectin-3 inhibitor. In certain embodiments, a dose of modified citrus pectin is from about 1 g to about 10 g, from about 3 g to about 7 g, or about 5 g

In certain embodiments, the galectin-3 inhibitor is administered enterally or parenterally, e.g., oral, sublingual, rectal, intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, etc., or combinations thereof.

In certain embodiments, the galectin-3 inhibitor is administered orally to the subject. In certain embodiments, the galectin-3 inhibitor is formulated in the form of a nutritional therapy. In certain embodiments, the galectin-3 inhibitor is formulated in the form of a supplement.

In certain embodiments, the galectin-3 inhibitor is administered to the eye of the subject. In certain embodiments, the galectin-3 inhibitor is administered externally to the eye of the subject. In certain embodiments, the galectin-3 inhibitor is administered to the subject intraocularly. In certain embodiments, the galectin-3 inhibitor is administered to the subject using a sustained release ocular delivery device. In certain embodiments, the galectin-3 inhibitor is administered to the subject as an eye drop.

3. Patient Populations That May Derive Particular Benefits from the Therapeutic Methods

The method may be further characterized according to the subject suffering from retinal nerve damage due to glaucoma. For example, in certain embodiments, the subject is a human. In certain embodiments, the subject is an adult human.

In certain embodiments, the subject has a concentration of galectin-3 in a bodily fluid that is greater than the average concentration of galectin-3 in the same bodily fluid of a healthy subject. In certain embodiments, the bodily fluid is blood plasma. In certain embodiments, the bodily fluid is blood serum. In certain embodiments, the bodily fluid is intraocular fluid. In certain embodiments, the concentration of galectin-3 in a bodily fluid of the subject is at least 5%, 10%, 15%, 20%, 25%, 30%, 35%, 40%, 45%, 50%, 55%, 60%, 70%, 75%, 80%, 85%, 90%, 95%, or 100% greater than the average concentration of galectin-3 in the same bodily fluid of a healthy subject.

In certain embodiments, the subject features a concentration of galectin-3 in a bodily fluid that increases over time. To illustrate, in certain embodiments the subject has a concentration of galectin-3 in a bodily fluid that is greater than the concentration of galectin-3 in the same type of bodily fluid observed in the subject 1, 2, 3, 4, 5, 6, 7, 10, 12, or 14 days prior. In certain embodiments, the bodily fluid is blood plasma. In certain embodiments, the bodily fluid is blood serum. In certain embodiments, the bodily fluid is intraocular fluid. In certain embodiments, the concentration of galectin-3 in a bodily fluid of the subject is at least 5%, 10%, 15%, 20%, 25%, 30%, 35%, 40%, 45%, 50%, 55%, 60%, 70%, 75%, 80%, 85%, 90%, 95%, or 100% greater than the concentration of galectin-3 in the same type of bodily fluid observed in the subject 1, 2, 3, 4, 5, 6, 7, 10, 12, or 14 days prior.

In certain embodiments, the subject exhibits elevated intraocular pressure. To illustrate, in certain embodiments, the subject has an intraocular pressure that is greater than the intraocular pressure observed in a health subject. In certain embodiments, the intraocular pressure of the subject is at least 5%, 10%, 15%, 20%, 25%, 30%, 35%, 40%, 45%, 50%, 55%, 60%, 70%, 75%, 80%, 85%, 90%, 95%, or 100% greater than the average intraocular pressure of a healthy subject.

In certain embodiments, the subject suffers from glaucoma and exhibits one or more symptoms typical of glaucoma. In certain embodiments, the subject exhibits one or more symptoms of glaucoma including, but not limited to, eye pain, eye redness, nausea, vomiting, headache, blurred vision, tunnel vision, patchy blind spots in visual field, and seeing halos around lights.

In certain embodiments, the subject has been diagnosed with retinal nerve damage.

4. Therapeutic Improvements & Other Characteristics

The method may be further characterized according to the therapeutic benefit of administration of the galectin-3 inhibitor to the subject. For example, in certain embodiments, the method produces at least a 5%, 10%, 20%, 30%, 40%, 50%, 60%, 70%, 80%, 90%, or 95% reduction in a symptom of the retinal nerve damage. In certain embodiments, the method achieves at least a 25% reduction in a symptom of retinal nerve damage. In certain embodiments, the method achieves at least a 50% reduction in a symptom of retinal nerve damage. In certain embodiments, the method achieves at least a 90% reduction in a symptom of retinal nerve damage. In certain embodiments, the symptom of retinal nerve damage is the volume of fibrotic retinal nerve tissue.

In certain embodiments, the method reduces the severity of one or more symptoms of glaucoma in the subject, including, but not limited to, eye pain, eye redness, nausea, vomiting, headache, blurred vision, tunnel vision, patchy blind spots in visual field, and seeing halos around lights. In certain embodiments, the method produces at least a 5%, 10%, 20%, 30%, 40%, 50%, 60%, 70%, 80%, 90%, or 95% reduction in the severity or frequency of one or more symptoms of glaucoma in the subject.

In certain embodiments, the method may be further characterized according to the nature of the retinal nerve damage to be prevented and/or treated. In certain embodiments, the retinal nerve damage comprises damage to nerve endings from the optical nerve, wherein the nerve endings are located in the retina. In certain embodiments, the retinal nerve damage is damage to nerve endings from the optical nerve, wherein the nerve endings are located in the retina.

5. Combination Therapy

Another aspect of the invention provides for combination therapy. Galectin-3 inhibitors described herein may be used in combination with additional therapeutic agents to treat retinal nerve damage in a subject suffering from glaucoma. Additionally, galectin-3 inhibitors described herein may be used in combination with additional therapeutic agents for the prophylaxis of retinal nerve damage in a subject suffering from glaucoma.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

One or more additional therapeutic agents may be administered separately from a compound or composition of the invention, as part of a multiple dosage regimen. Alternatively, one or more additional therapeutic agents may be part of a single dosage form, mixed together with a compound of this invention in a single composition. If administered as a multiple dosage regime, one or more additional therapeutic agents and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more additional therapeutic agents and a compound or composition of the invention are administered as a multiple dosage regimen more than 24 hours apart.

As used herein, the term “combination,” “combined,” and related terms refers to the simultaneous or sequential administration of therapeutic agents in accordance with this invention. For example, a compound of the present invention can be administered with one or more additional therapeutic agent(s) simultaneously or sequentially in separate unit dosage forms or together in a single unit dosage form. Accordingly, the present invention provides a single unit dosage form comprising a compound of the current invention, one or more additional therapeutic agent(s), and a pharmaceutically acceptable carrier, adjuvant, or vehicle.

The amount of a compound of the invention and one or more additional therapeutic agent(s) (in those compositions which comprise an additional therapeutic agent) that can be combined with the carrier materials to produce a single dosage form varies depending upon the host treated and the particular mode of administration. Preferably, a composition of the invention should be formulated so that a dosage of between 0.01-100 mg/kg body weight/day of a compound of the invention can be administered.

In those compositions which comprise one or more additional therapeutic agent(s), the one or more additional therapeutic agent(s) and a compound of the invention can act synergistically. Therefore, the amount of the one or more additional therapeutic agent(s) in such compositions may be less than that required in a monotherapy utilizing only that therapeutic agent. In such compositions a dosage of between 0.01-1,000 g/kg body weight/day of the one or more additional therapeutic agent(s) can be administered.

The amount of one or more additional therapeutic agent(s) present in the compositions of this invention is preferably no more than the amount that would normally be administered in a composition comprising that therapeutic agent as the only active agent. Preferably the amount of one or more additional therapeutic agent(s) in the presently disclosed compositions ranges from about 50% to 100% of the amount normally present in a composition comprising that agent as the only therapeutically active agent. In some embodiments, one or more additional therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA-approved therapeutic agent is approved for dosing per the FDA label insert.

In certain embodiments, the method further comprises administering an additional therapeutic agent.

In certain embodiments, the additional therapeutic agent is an agent that reduces intraocular pressure. In certain embodiments, the additional therapeutic agent is an anti-glaucoma medicine.

In certain embodiments, the additional therapeutic agent is
A prostaglandin analog, such as latanoprost, bimatoprost, travoprost, tafluprost, latanoprostene bunod, or a pharmaceutically acceptable salt thereof;
A beta blocker, such as timolol or a pharmaceutically acceptable salt thereof;
An alpha agonist, such as brimonidine or a pharmaceutically acceptable salt thereof;
A carbonic anhydrase inhibitor, such as dorzolamide, brinzolamide, acetazolamide, methazolamide, or a pharmaceutically acceptable salt thereof;
A cholinergic agonist, such as pilocarpine or a pharmaceutically acceptable salt thereof; or
A Rho kinase inhibitor, such as netarsudil or a pharmaceutically acceptable salt thereof.

In certain embodiments, the subject has undergone surgery to install a tube into the eye to reduce intraocular pressure.

6. Additional Optional Steps

The method may be further characterized according to one or more additional steps carried out as part of the method. For example, in certain embodiments, the method further comprises determining if the subject has glaucoma. Diagnosis of glaucoma in the subject can be carried out through use of one or more tests including, but not limited to, measurement of intraocular pressure (tonometry), dilated eye examination and imaging, visual field test, measurement of corneal thickness (pachymetry) and inspection of the drainage angle (gonioscopy).

In certain embodiments, the method further comprises determining if the subject has retinal nerve damage due to elevated intraocular pressure.

In certain embodiments, the method further comprises measuring the subject's blood plasma levels of galectin-3. In certain embodiments, the method further comprises measuring the subject's intraocular levels of galectin-3.

In certain embodiments, the method further comprises monitoring subject response to the galectin-3 inhibitor to evaluate therapeutic benefit. In certain embodiments, the evaluation of the therapeutic benefit comprises an evaluation of one or more of the therapeutic improvements described above.

### II. Compositions for Medical Use

Galectin-3 inhibitors described herein may be used to prevent and treat retinal nerve damage in a subject suffering from glaucoma, as described above. The use may be according to a method described herein. For example, one aspect of the invention provides a galectin-3 inhibitor for use in treating retinal nerve damage in a subject suffering from glaucoma. Another aspect of the invention provides a galectin-3 inhibitor for use in slowing the progression of retinal nerve damage in a subject suffering from glaucoma. Another aspect of the invention provides a galectin-3 inhibitor for use in reducing the risk of retinal nerve damage in a patient suffering from glaucoma. Another aspect of the invention provides a galectin-3 inhibitor for use in preventing the development of retinal nerve damage in a patient suffering from glaucoma.

Embodiments described herein in connection with the methods for treatment may be applied in connection with the galectin-3 inhibitors for use.

### III. Preparation of a Medicament

Galectin-3 inhibitors described herein may be used in the preparation of a medicament to prevent and treat retinal nerve damage in a subject suffering from glaucoma, as described above. For example, one aspect of the invention provides for the use of a galectin-3 inhibitor described herein in the preparation of a medicament for treating retinal nerve damage in a subject suffering from glaucoma. Another aspect of the invention provides for the use of a galectin-3 inhibitor described herein in the preparation of a medicament for slowing the progression of retinal nerve damage in a subject suffering from glaucoma. Another aspect of the invention provides for the use of a galectin-3 inhibitor described herein in the preparation of a medicament for reducing the risk of retinal nerve damage in a patient suffering from glaucoma. Another aspect of the invention provides for the use of a galectin-3 inhibitor described herein in the preparation of a medicament for preventing the development of fibrotic retinal nerve tissue resulting from glaucoma.

Embodiments described herein in connection with the methods for treatment may be applied in connection with the galectin-3 inhibitors for use in the preparation of a medicament.

### IV. Pharmaceutical Compositions

As indicated above, the invention provides pharmaceutical compositions, which comprise a compound described above and a pharmaceutically acceptable carrier. The pharmaceutically acceptable carrier may contain additive(s) and/or diluent(s). The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In certain embodiments, a formulation of the invention suitable for oral administration is formulated in the form of a nutritional therapy. In certain embodiment, a formulation of the invention suitable for oral administration is formulated in the form of a supplement.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising a compound described herein in a therapeutically effective amount for the treatment of a medical disorder described herein.

### V. Kits

Another aspect of the invention provides a medical kit comprising, for example, (i) a galectin-3 inhibitor, and (ii) instructions for use according to a method described herein (e.g., treating retinal nerve damage in a patient suffering from glaucoma according to a method described herein).

### EXAMPLES

The invention now being generally described, will be more readily understood by reference to the following examples, which are included merely for purposes of illustration of certain aspects and embodiments of the present invention, and are not intended to limit the invention.

### Example 1

### Analysis of Galectin-3 Levels in Microglia in Mice with Glaucoma

Galectin-3 levels in microglia were analyzed in mice suffering from glaucoma. Experimental procedures and results are provided below.

### Part I—Experimental Procedure

Mice: C57BL/6J (wildtype) and B6.129P2-ApoetmlUnc/J (Apoe^{-/-}) mice were purchased from JAX. Mice were a mix of both genders and 6-12 weeks of age at the beginning of the experiments. Mice were housed under specific pathogen free conditions with food and water ad libitum. Mice did not undergo any procedures prior to their stated use. Mice were euthanized by CO₂ inhalation. The Institutional Animal Care and Use Committee at Harvard Medical School, Brigham and Women's Hospital, and MEEI Schepens Eye Research Institute approved all experimental procedures involving animals.

Magnetic Microbead Injection: Microbead injection was performed based on the procedure in Sappington RM, et al. in Invest Ophthalmol Vis Sci (2010) 51(1):207-216; Chen H, et al. in Invest Ophthalmol Vis Sci (2011) vol. 52(1):36-44; and Ito YA, et al. in J Vis Exp (2016) vol. 109:e53731. Briefly, mice were anesthetized by i.p. injection of a mixture of ketamine (100 mg kg⁻¹) and xylazine (10 mg kg⁻¹) and pupils dilated with 1% tropicamide. A small puncture was made in the cornea using a 30-gauge needle. Eyes were injected with 1.5 μL of magnetic microbead solution (2.4×10⁶ beads) or PBS for sham injections. All injections were done in the left eye. Beads were attracted to and evenly distributed around the anterior chamber using a small magnet, and eyes were treated with antibiotic eyedrops to reduce risk of infection. Intraocular pressure was monitored as described below.

IOP Measurement: IOP was measured 24 hours after the microbead injection, and then twice a week using a tonometer (TonoLab; Icare, Finland). Mice were anesthetized by isoflurane inhalation (2% to 4% flow). Measurements were conducted at consistent times in the morning and were performed for 1 month following the microbead injection. The tonometer records six measurements after excluding outlying values and displays an average. The tonolab-generated average was considered one value, and we recorded five values per eye. The mean of these five values determined the IOP measurement.

Mouse microglia isolation and sorting: Microglia isolation was performed according to the protocol in Butovsky O, et al. in Nat Neurosci (2014) vol. 17(1):131-143 and Krasemann S, et al. in Immunity (2017) vol. 47(3):566-581 e569. Briefly, mice were euthanized using CO₂, eyes removed, and retinas dissected. Single cell suspensions were prepared and centrifuged over a 37%/70% discontinuous Percoll gradient (GE Healthcare), and mononuclear cells were isolated from the interface. In order to distinguish resident microglia from recruited myeloid cells, we used a monoclonal antibody that recognizes Fcrls, which is expressed on microglia but not on infiltrating myeloid cells. See Butovsky O, et al. Nat Neurosci (2014) vol. 17(1):131-143, Isolated cells were stained with anti-Fcrls [clone 4G11, 3 μg ml⁻¹, Butovsky lab, validated in references Butovsky O, et al. Nat Neurosci (2014) vol. 17(1):131-143 and Butovsky O, et al. Ann Neurol (2015) vol. 77(1):75-99. CD11b-PeCy7 [clone M1/70, BD Biosciences, 2 μg ml⁻¹] and Ly6c-PerCP/Cy5.5 [clone HK1.4, BioLegend, 2 μg ml⁻¹] antibodies to specifically sort resident microglia as CD11b⁺, Ly6c⁻, and Fcrls⁺cells. 100 to 600 cells were collected from each retina, normalized to 100 cells per 5 μl of TCL buffer, and submitted for RNAseq.

Preparation of primary neuron culture and induction of apoptosis: For detailed description of isolation of primary neurons, induction of apoptosis and labeling of neurons, see Krasemann S, et al. Immunity (2017) vol. 47(3):566-581 e569. Briefly, neurons were isolated and cultured from mouse embryos at age E18.5. 7-10 days after culture, neurons were detached from culture plates and apoptosis was induced by UV irradiation with an intensity of 6×15 W for 15 min. Neurons were then labeled with fluorescent dye (Alexa488 5-SDP Ester or Alexa405 NHS Ester, Life Technologies/Thermo Fisher Scientific) and total apoptotic cell number was determined using Trypan Blue staining. Neurons were resuspended at a density of approximately 25,000 cells per μl.

Injection of apoptotic neurons into the eye and isolation of phagocytic vs. non-phagocytic microglia: Apoptotic neurons were prepared as described above. Recipient mice were anesthetized by i.p. injection of ketamine (100 mg kg⁻¹) and xylazine (10 mg kg⁻¹) and pupils were dilated with 1% tropicamide. A 30-gauge needle was used to perform the initial puncture just posterior to the limbus, with care being taken to avoid injuring the lens. 2 μl of labeled apoptotic neurons were loaded into a 10 μl. Nanofil microsyringe and injected intravitreally. 16 to 24 hours after the injection, mice were sacrificed by CO₂ inhalation. Microglia isolation was performed according to the protocol in Butovsky O, et al. Nat Neurosci (2014) vol. 17(1):131-143). Briefly, eyes were removed from mice, retinas dissected and microglia isolated by using FACS sorting as described above. 5-6 retinas were pooled to create each sample in order to increase the yield of phagocytic microglia (100-500 phagocytic cells per sample). Microglia were isolated as CD11b⁺, Ly6c⁻, and Fcrls+cells, and phagocytic versus non-phagocytic microglia were further sorted from the Fcrls⁺CD11b⁺-population by detection of Alexa488 or Alexa405 fluorescence.

RNA sequencing: Samples were processed according to the Smart-Seq2 protocol and sequenced on Illumina sequencers. Reads in FASTQ were quantified at the transcript level using Salmon against an Ensembl catalog and aggregated to the gene level using tximport. Data were analyzed using t-test (for datasets with two experimental groups) or 1-way ANOVA with Tukey's posthoc test (for datasets with multiple experimental groups) with a set at 0.05.

Immunohistochemistry: Following euthanasia, eyes were enucleated and fixed in 4% PFA for 24 hours at 4° C. For Ibal/Gal-3 staining, retinas were dissected from enucleated eyes and immediately transferred to ice-cold methanol and kept in methanol for 20 minutes on ice. Next, retinas were washed with PBS 0.3% triton, permeabilized by freezing at −80° C. for 15 minutes in PBS 03% triton, and washed once more with PBS 0.3% triton. Retinas were blocked for 1 hour at RT in blocking buffer (5% NHS, 0.2% BSA, 0.3% Triton-X100 in 1× PBS). After blocking, retinas were incubated with anti-Gal-3 antibody (monoclonal, clone B2C10, #556904, BD Pharmingen, 0.5 μg ml⁻¹;1:200) and anti-Ibal antibody (polyclonal, #019-19741, WAKO Chemicals, 1 μg ml⁻¹; 1:200) overnight at 4° C. After primary antibody incubation, retinas were washed with PBS 0.3% triton and incubated with AlexaFluor 594 goat anti-mouse (polyclonal, #A11005, Invitrogen; 1:400) and AlexaFluor 488 chicken anti-rabbit (cross adsorbed, #A21441, Invitrogen; 1:400) secondary antibodies in blocking buffer for 2 hours at room temperature. Retinas were then washed in 1× PBS, stained with DAN, and mounted on microscope slides vitreous side up using VectaSheld with DAPI mounting medium.

### Part II—Results

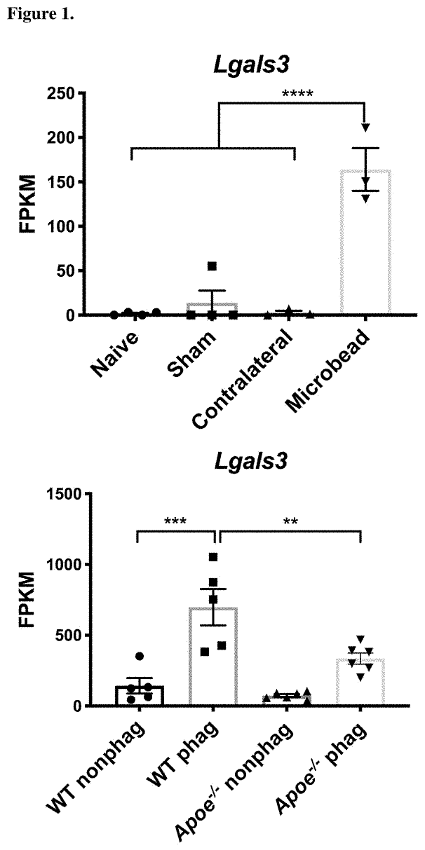

Results of the experiment show that galactin-3 was upregulated at the mRNA level in mice suffering from glaucoma, but that galactin-3 was not upregulated at the mRNA level in control mice (i.e., mice not suffering from glaucoma). Furthermore, galectin-3 was upregulated in retinal microglia in response to apoptotic neurons. This upregulation was attenuated in APOE knock-out mice, showing that galectin-3 is a downstream effector of APOE. APOE is a known regulator of the neurodegeneration-associated microglial phenotype that exacerbates retinal ganglion cell degeneration in glaucoma. The results are displayed graphically in FIG. 1.

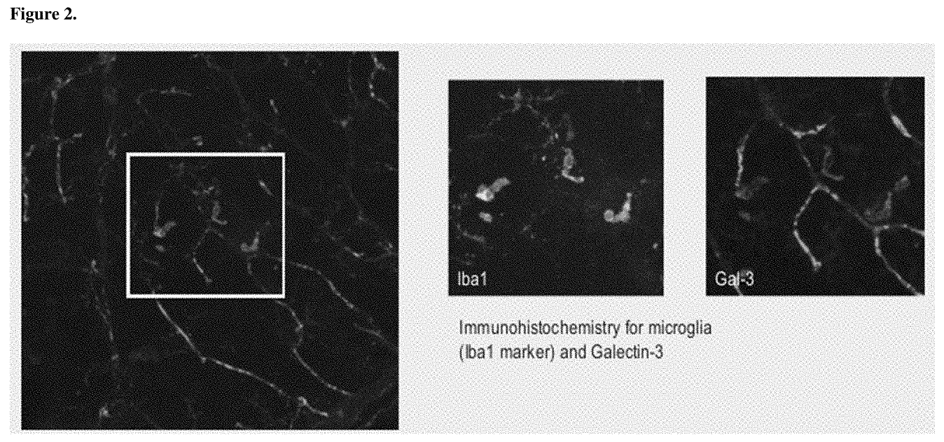

Additionally, results of the experiment show that galactin-3 was upregulated at the protein level in microglia in mice suffering from glaucoma. The results are displayed graphically in FIG. 2.

### Example 2

### Analysis of Galectin-3 Levels in Microglia in APOE Knockout Mice Suffering from Glaucoma

Galectin-3 levels in microglia were analyzed in wildtype and APOE knockout mice suffering from glaucoma. Experimental procedures and results are provided below.

### Part I—Experimental Procedure

Magnetic Microbead Injection: Microbead injection was performed based on the procedure in Sappington R M, et al. in Invest Ophthalmol Vis Sci (2010) 51(1):207-216; Chen H, et al. in Invest Ophthalmol Vis Sci (2011) vol. 52(1):36-44; and Ito YA, et al. in J Vis Exp (2016) vol. 109:e53731. Briefly, mice were anesthetized by i.p. injection of a mixture of ketamine (100 mg kg⁻¹) and xylazine (10 mg kg⁻¹) and pupils dilated with 1% tropicamide. A small puncture was made in the cornea using a 30-gauge needle. Eyes were injected with 1.5 μL of magnetic microbead solution (2.4×10⁶ beads) or PBS for sham injections. All injections were done in the left eye. Beads were attracted to and evenly distributed around the anterior chamber using a small magnet, and eyes were treated with antibiotic eyedrops to reduce risk of infection. Intraocular pressure was monitored as described below.

IOP Measurement: IOP was measured 24 hours after the microbead injection, and then twice a week using a tonometer (TonoLab; Icare, Finland). Mice were anesthetized by isoflurane inhalation (2% to 4% flow). Measurements were conducted at consistent times in the morning and were performed for 1 month following the microbead injection. The tonometer records six measurements after excluding outlying values and displays an average. The tonolab-generated average was considered one value, and we recorded five values per eye. The mean of these five values determined the IOP measurement.

Immunohistochemistry: Following euthanasia, eyes were enucleated and fixed in 4% PFA for 24 hours at 4° C. For P2ryl2/Gal-3 staining, retinas were dissected from enucleated eyes and immediately transferred to ice-cold methanol and kept in methanol for 20 minutes on ice. Next, retinas were washed with PBS 0.3% triton, permeabilized by freezing at −80° C. for 15 minutes in PBS 0.3% triton, and washed once more with PBS 0.3% triton. Retinas were blocked for 1 hour at RT in blocking buffer (5% NHS, 0.2% BSA, 0.3% Triton-X100 in 1× PBS). After blocking, retinas were incubated with either anti-Gal-3 antibody (monoclonal, clone B2C10, #556904, BD Pharmingen, 0.5 pg ml⁻¹;1:200) and anti-P2ryl2 [polyclonal, 0.4 g ml⁻¹, Butovsky lab, validated in references Butovsky O, et al. in Nat Neurosci (2014) vol. 17(1):131-143 and Butovsky O, et al. in Ann Neurol (2015) vol. 77(1):75-99; 1:200] overnight at 4° C. After primary antibody incubation, retinas were washed with PBS 0.3% triton and incubated with AlexaFluor 594 goat anti-mouse (polyclonal, #A11005, Invitrogen; 1:400) and AlexaFluor 488 chicken anti-rabbit (cross adsorbed, #A21441, Invitrogen; 1:400) secondary antibodies in blocking buffer for 2 hours at room temperature. Retinas were then washed in 1× PBS, stained with DAPI, and mounted on microscope slides vitreous side up using VectaSheld with DAPI mounting medium.

### Part II—Results

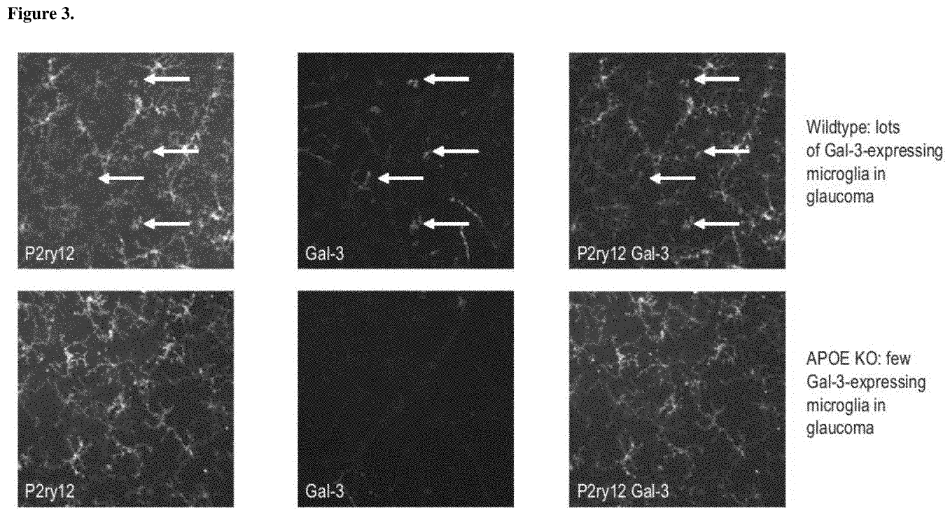

Results of the experiment show that galactin-3 expression is attenuated in microglia in APOE knockout mice suffering from glaucoma relative to galactin-3 expression in wildtype mice suffering from glaucoma. APOE is a known regulator of the neurodegeneration-associated microglial phenotype that exacerbates retinal ganglion cell degeneration in glaucoma. This result shows that galectin-3 is a downstream effector of APOE in microglia. The results are displayed graphically in FIG. 3.

### Example 3

### Analysis of Retinal Ganglion Cell Survival in Galectin-3 Knockout Mice with Glaucoma

The number of surviving retinal ganglion cells was analyzed in galectin-3 knockout mice suffering from glaucoma as compared to wildtype animals with glaucoma and controls without glaucoma. Experimental procedures and results are provided below.

### Part I—Experimental Procedure

Mice: C57BL/6J (wildtype) and 6.Cg-Lgals3 tm1Poi/J (Lgals3^{-/-}) mice were purchased from JAX. Mice were a mix of both genders and 6-12 weeks of age at the beginning of the experiments. Mice were housed under specific pathogen free conditions with food and water ad libitum. Mice did not undergo any procedures prior to their stated use. Mice were euthanized by CO₂ inhalation. The Institutional Animal Care and Use Committee at Harvard Medical School, Brigham and Women's Hospital, and MEET Schepens Eye Research Institute approved all experimental procedures involving animals.

Magnetic Microbead Injection: Microbead injection was performed based on the procedure in Sappington R M, et al. in Invest Ophthalmol Vis Sci (2010) 51(1):207-216; Chen H, et al. in Invest Ophthalmol Vis Sci (2011) vol. 52(1):36-44; and Ito YA, et al. in J Vis Exp (2016) vol. 109:e53731. Briefly, mice were anesthetized by i.p. injection of a mixture of ketamine (100 mg kg⁻¹) and xylazine (10 mg kg⁻¹) and pupils dilated with 1% tropicamide. A small puncture was made in the cornea using a 30-gauge needle. Eyes were injected with 1.5 μL of magnetic microbead solution (2.4×10⁶ beads) or PBS for sham injections. All injections were done in the left eye. Beads were attracted to and evenly distributed around the anterior chamber using a small magnet, and eyes were treated with antibiotic eyedrops to reduce risk of infection. Intraocular pressure was monitored as described below.

IOP Measurement: IOP was measured 24 hours after the microbead injection, and then twice a week using a tonometer (TonoLab; Icare, Finland). Mice were anesthetized by isoflurane inhalation (2% to 4% flow). Measurements were conducted at consistent times in the morning and were performed for 1 month following the microbead injection. The tonometer records six measurements after excluding outlying values and displays an average. The tonolab-generated average was considered one value, and we recorded five values per eye. The mean of these five values determined the TOP measurement.

Immunohistochemistry: Following euthanasia, eyes were enucleated and fixed in 4% PFA for 24 hours at 4° C. For Bm3a staining used for RGC quantification, retinas were dissected and permeabilized in PBS 0.3% triton by freezing for 15 minutes at −80° C. Next, the retinas were rinsed with PBS, blocked in blocking buffer for 1 hour at room temperature, rinsed with 1× PBS, and incubated with anti-Brn3a (monoclonal, #MAB1585, Millipore; 1:200) in blocking buffer for 4-6 days at 4° C. Retinas were washed 3× in 1× PBS at RT and incubated with AlexaFluor 594 goat anti-mouse secondary antibody (polyclonal, #A11005, Invitrogen; 1:400) in blocking buffer for 2 days at 4° C. Following secondary incubation, retinas were washed in 1× PBS and slides were prepared.

Retinal Ganglion Cell Quantification: Flatmounted retinas were imaged using Zeiss LSM 710 Confocal Microscope. Images were taken at 63X using an oil immersion objective, and 12 images were collected per sample (3 images per quadrant in retinal mid-periphery). Brn3a⁺ DAPI⁺ double positive cells were manually counted using ImageJ. RGC count per sample was averaged over the 12 images and converted to cells/mm².

### Part II—Results

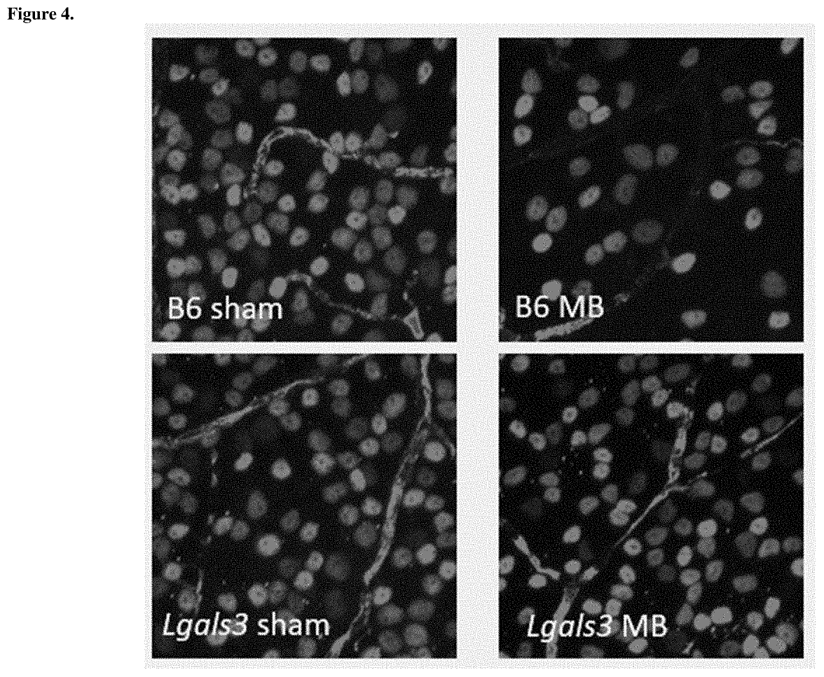

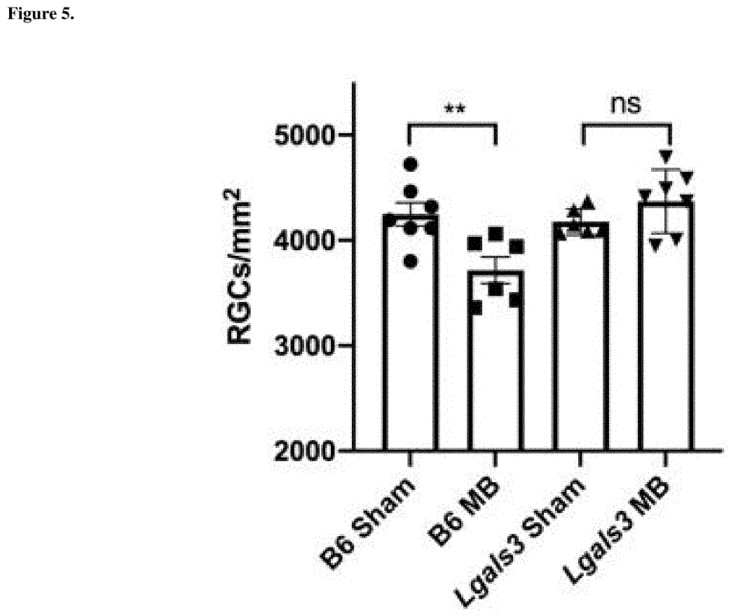

Results of the experiment show that in wildtype animals retinal ganglion cell numbers decreased in animals with glaucoma compared to control. In contrast, in galectin-3 knockout mice, retinal ganglion cells survived despite elevated intraocular pressure. Therefore, genetic targeting of galectin-3 is neuroprotective in the mouse model of glaucoma. The results are displayed graphically in FIGS. 4 and 5.

### Example 4

### Analysis of Ability of Galectin-3 Inhibitors to Protect Against Retinal Ganglion Cell Loss in Mice Suffering from Glaucoma

The ability of galectin-3 inhibitors to protect against loss of retinal ganglion cells in mice suffering from glaucoma can be evaluated according to the experimental procedures provided below.

Part I—Experimental Procedure

Magnetic Microbead Injection: Microbead injection can be performed based on the procedure in Sappington R M, et al. in Invest Ophthalmol Vis Sci (2010) 51(1):207-216; Chen H, et al. in Invest Ophthalmol Vis Sci (2011) vol. 52(1):36-44; and Ito YA, et al. in J Vis Exp (2016) vol. 109:e53731. Briefly, mice are anesthetized by i.p. injection of a mixture of ketamine (100 mg kg⁻¹) and xylazine (10 mg kg⁻¹) and pupils dilated with 1% tropicamide. A small puncture is made in the cornea using a 30-gauge needle. Eyes are injected with 1.5 μL of magnetic microbead solution (2.4×10⁶ beads) or PBS for sham injections. All injections are done in the left eye. Beads are attracted to and evenly distributed around the anterior chamber using a small magnet, and eyes are treated with antibiotic eyedrops to reduce risk of infection. Intraocular pressure can be monitored as described below.

IOP Measurement: TOP is measured 24 hours after the microbead injection, and then twice a week using a tonometer (TonoLab; Icare, Finland). Mice are anesthetized by isoflurane inhalation (2% to 4% flow). Measurements are conducted at consistent times in the morning and were performed for 1 month following the microbead injection. The tonometer records six measurements after excluding outlying values and displays an average. The tonolab-generated average is considered one value, and may be recorded five values per eye. The mean of these five values determines the TOP measurement.

Injection of galectin-3 inhibitor in glaucomatous eyes: Galectin-3 inhibitor (such as TD139, #28400, CAS #1450824-22-2, Cayman Chemicals; C₂₈H₃₀F₂N₆O₈S) is prepared by dissolving the solid compound in DMSO to 1000 ng μl¹, then further diluting this stock in 1× PBS to a final concentration of 50 ng μl⁻¹ in 5% DMSO/PBS. At two timepoints (2 and 3 weeks after microbead injection), wildtype mice are anesthetized as described above and administered 1 μl of galectin-3 inhibitor or 5% DMSO/PBS vehicle in the microbead-injected eye via intravitreal injection. Sham-injected eyes are treated intravitreally with vehicle. At 4 weeks after microbead injection, mice are sacrificed by CO₂ inhalation and eyes are collected for immunohistochemistry and RGC quantification. Toxicity is assessed prior to the experiment to confirm that retinal ganglion cell density is not affected by galectin-3 inhibitor treatment under control conditions.

Immunohistochemistry: Following euthanasia, eyes are enucleated and fixed in 4% PFA for 24 hours at 4° C. For Brn3a staining used for RGC quantification, retinas are dissected and permeabilized in PBS 0.3% triton by freezing for 15 minutes at −80° C. Next, the retinas are rinsed with PBS, blocked in blocking buffer for 1 hour at room temperature, rinsed with 1× PBS, and incubated with anti-Brn3a (monoclonal, #MAB1585, Millipore; 1;200) in blocking buffer for 4-6 days at 4° C. Retinas are washed 3× in 1× PBS at RT and incubated with AlexaFluor 594 goat anti-mouse secondary antibody (polyclonal, #A11005, Invitrogen; 1:400) in blocking buffer for 2 days at 4° C. Following secondary incubation, retinas are washed in 1× PBS and slides were prepared.

Retinal Ganglion Cell (RGC) Quantification: Flatmounted retinas may be imaged using Zeiss LSM 710 Confocal Microscope. Images are taken at 63X using an oil immersion objective, and 12 images are collected per sample (3 images per quadrant in retinal mid-periphery). Brn3a⁺ DAPI⁺ double positive cells may be manually counted using ImageJ. RGC count per sample may be averaged over the 12 images and converted to cells/mm².

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating retinal nerve damage in a subject suffering from glaucoma, comprising administering to the subject in need thereof a therapeutically effective amount of a galectin-3 inhibitor to treat the retinal nerve damage.

2. The method of claim 1, wherein the method achieves at least a 25% reduction in the volume of retinal nerve damage.

3. The method of claim 1, wherein the method achieves at least a 50% reduction in the volume retinal nerve damage.

4. A method for the prophylaxis of retinal nerve damage in a subject suffering from glaucoma, comprising administering to the subject in need thereof a therapeutically effective amount of a galectin-3 inhibitor.

5. A method of reducing the risk of retinal nerve damage in a subject suffering from glaucoma, comprising administering to a subject in need thereof an effective amount of a galectin-3 inhibitor to reduce the risk of retinal nerve damage in the subject.

6. The method of claim 5, further comprising determining if the subject has glaucoma.

7. The method of claim 5, further comprising determining if the subject has retinal nerve damage due to elevated intraocular pressure.

8. The method of claim 5, further comprising selecting the subject to receive the galectin-3 inhibitor if the patient has been diagnosed with retinal nerve damage.

9. The method of claim 1, further comprising determining if the subject's eye has elevated intraocular pressure.

10. The method of claim 1, further comprising selecting the subject to receive the galectin-3 inhibitor if the patient has elevated blood plasma levels of galectin-3.

11. The method of claim 1, further comprising selecting the subject to receive the galectin-3 inhibitor if the patient has elevated intraocular levels of galectin-3.

12. The method of claim 1, further comprising monitoring subject response to the galectin-3 inhibitor to evaluate therapeutic benefit.

13. (canceled)

14. The method of claim 1, wherein the galectin-3 inhibitor is an oligosaccharide.

15. The method of claim 14, wherein the oligosaccharide is a neo-glycan.

16. The method of claim 14, wherein the oligosaccharide is N-acetyllactosamine.

17. (canceled)

18. The method of claim 14, wherein the oligosaccharide is N,N-diacetyllactosamine.

19. (canceled)

20. (canceled)

21. The method of claim 1, wherein the galectin-3 inhibitor is a polysaccharide.

22. The method of claim 1, wherein the galectin-3 inhibitor comprises galactose.

23. The method of claim 1, wherein the galectin-3 inhibitor comprises a mixture of pectic fragments.

24. The method of claim 1, wherein the galectin-3 inhibitor comprises a pectin-derived moiety.

25. The method of claim 1, wherein the galectin-3 inhibitor is a pectin.

26. The method of claim 1, wherein the galectin-3 inhibitor is a modified citrus pectin.

27. The method of claim 1, wherein the galectin-3 inhibitor is a pumpkin pectin.

28. The method of claim 1, wherein the galectin-3 inhibitor is an antibody.

29. The method of claim 1, wherein the subject is an adult human.

30. (canceled)

31. (canceled)

32. (canceled)

33. The method of claim 1, wherein the galectin-3 inhibitor is administered to the eye.

34-40. (canceled)
